(19) United States
(12) Patent Application Publication
(10) Pub. No.: US 2022/0369993 A1
(43) Pub. Date: Nov. 24, 2022

(54) Methods And Systems For Placement Of An Electrode Array During An Intracranial Electroencephalogram

(71) Applicant: Stryker Corporation, Kaamazoo, MI (US)

(72) Inventor: Allison Bolthouse, Mattawan, MI (US)

(73) Assignee: Stryker Corporation, Kalamazoo, MI (US)

(21) Appl. No.: 17/774,687

(22) PCT Filed: Nov. 6, 2019
(86) PCT No.: PCT/US2020/059340

§ 371 Date: May 5, 2022

Related U.S. Application Data
(60) Provisional application No. 62/931,551, filed on Nov. 6, 2019.

(51) Int. Cl. A61B 5/369; A61B 5/00

(52) U.S. Cl. CPC A61B 5/369; A61B 5/7264; A61B 5/742

(57) ABSTRACT

An electrode system for an intracranial electroencephalogram is described. A substrate is configured to be applied directly on a brain of a patient. The substrate has a profile that is suitable to be placed within a subdural space in between a portion of intact cranium and cerebral cortex of the brain. A tracking device is arranged on the substrate. A plurality of electrodes are configured to sense electrical activity associated with the brain. Each of the plurality of electrodes has a predefined relationship with the tracking device. A tracking system is operable to determine a position of the tracking device relative to a reference point based on a strength of an electromagnetic field relative to the tracking device. A controller is configured to selectively determine a position for each of the plurality of electrodes based on the position of the tracking device and the predefined relationship.

### CROSS-REFERENCE TO RELATED APPLICATIONS

The subject patent application claims priority to and all of the benefits of U.S. Provisional Patent Application No. 62/931,551, filed on Nov. 6, 2019, the disclosure of which is hereby incorporated by reference.

### BACKGROUND

Epilepsy is a neurological disorder in which the normal pattern of electrical activity is disrupted. The disruption of electrical activity may cause recurrent seizures. It is common for a patient with refractory epilepsy to undergo surgical resection to resect epileptogenic tissue. In order to identify or confirm a region of interest for surgical resection, tests may be performed. One such test that is commonly performed is an intracranial electroencephalogram (iEEG) also known as an electrocorticogram, in which a subdural electrode array is placed directly on the brain of the patient in order to monitor the electrical activity of the brain. The subdural electrode array may remain implanted for a short period of time (i.e., several hours) or an extended period of time (i.e., several days). After the measurements and data from the iEEG are analyzed, and a problem area is identified, resection surgery may be performed in order to resect all or part of the problem area.

The background description provided here is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### SUMMARY

Further areas of applicability of the present disclosure will become apparent from the detailed description, the claims, and the drawings. The detailed description and specific examples are intended for purposes of illustration only and are not intended to limit the scope of the disclosure.

In a feature, an electrode system for at least one of an intracranial electroencephalogram, an intracranial electroencephalography, and an electrocorticography, the electrode system is described. An electrode array includes a substrate, a tracking device, and a plurality of electrodes. The substrate is configured to be applied directly on a brain of a patient. The substrate is conformable to a shape of the brain and has a profile that is suitable to be placed within a subdural space in between a portion of intact cranium and cerebral cortex of the brain. The tracking device is arranged on the substrate. The plurality of electrodes are also arranged on the substrate and configured to sense electrical activity associated with the brain. Each of the plurality of electrodes has a predefined relationship with the tracking device. A tracking system is operable to determine a position of the tracking device relative to a reference point based on a strength of an electromagnetic field relative to the tracking device. A controller is configured to selectively determine a position for each of the plurality of electrodes based on the position of the tracking device and the predefined relationship.

In a feature, a method for tracking a location of a plurality of electrodes of an electrode array for performing at least one of an intracranial electroencephalogram, an intracranial electroencephalography, and an electrocorticography is described. The subdural electrode array includes a substrate with a tracking device and the plurality of electrodes. Each of the plurality of electrodes has a predefined relationship with the tracking device. The method includes sliding the subdural electrode array between at least one of: a portion of an intact cranium and a cortex of a patient; and on a cortical surface through an opening due to a bone flap. The method also includes selectively determining a position of the tracking device relative to a reference point, based on a strength of a generated electromagnetic field, relative to the tracking device. The method also includes determining a position for each of the plurality of electrodes based on the position of the tracking device.

In a feature, an electrode array for at least one of an intracranial electroencephalogram, an intracranial electroencephalography, and an electrocorticography is described. The subdural electrode array includes a substrate, a tracking device, and a plurality of electrodes. The substrate is configured to be applied directly on a brain of a patient. The substrate is conformable to a shape of the brain and has a profile suitable to be placed within a subdural space in between a portion of intact cranium and cerebral cortex of the brain. A tracking device is arranged on the substrate and is configured to generate a signal that is indicative of a position of the tracking device. A plurality of electrodes is configured to sense electrical activity of the brain. Each of the plurality of electrodes is disposed in a predefined relationship to the tracking device.

### BRIEF DESCRIPTION OF THE DRAWINGS

The present disclosure will become more fully understood from the detailed description and the accompanying drawings.

FIG. 1 is an example electrode system according to the teachings of the present disclosure.

FIG. 2 is an example subdural grid electrode array according to the teachings of the present disclosure.

FIG. 3 is an example subdural strip electrode array according to the teachings of the present disclosure.

FIG. 4 is a schematic of an example subdural grid electrode array applied to a brain of a patient according to the teachings of the present disclosure.

FIG. 5 a functional block diagram of an electrode system according to the teachings of the present disclosure.

FIG. 6 is a virtual representation of showing selected target areas of a brain of a patient according to the teachings of the present disclosure.

FIG. 7 is a virtual representation of a subdural grid electrode array and multiple subdural strip electrode arrays overlaid onto a brain of a patient according to the teachings of the present disclosure.

FIG. 8 shows multiple views of a virtual representation of a brain of a patient according to the teachings of the present disclosure.

FIG. 9 is an example flowchart performed by an electrode system according to the teachings of the present disclosure.

In the drawings, reference numbers may be reused to identify similar and/or identical elements.

### DETAILED DESCRIPTION

Prior to performing an iEEG, a craniotomy may be performed to remove part of the cranium in order to expose the brain. The removed part of the cranium may be referred to as a bone flap throughout this disclosure. A subdural electrode array is then placed directly on a cortical surface of the patient. For example, the subdural electrode array may be placed in a subdural space such as between a portion of an intact cranium and a cortex of the patient, where only a portion of the subdural electrode array is visible.

In present systems, intraoperative images (e.g., MRIs or CT scans) are captured after the subdural electrode array has been applied to the patient in order to confirm placement of the subdural electrode array since at least a portion of the subdural electrode array is placed in an area that does not have line of sight with a medical professional or a tracking system. Once the medical professional is satisfied with the placement of the subdural electrode array, the medical professional records the location of the subdural electrode array, including a location for each electrode of the array, on a schematic of the brain. Such systems are time consuming and subject to human error. Thus, systems and methods for assisting a surgeon with navigated placement of an electrode array are desirable. Such systems would reduce the possibility of human errors caused by improper recording of the location of the subdural electrode array and/or each of the electrodes. Such systems also offer a radiation-free method of real-time electrode visualization as opposed to systems that require intraoperative images. Lastly, such systems allow medical professionals to implant less electrodes since the medical professional knows the precise location of the subdural electrode array and does not need to implant many electrodes to guarantee vast coverage of a target area.

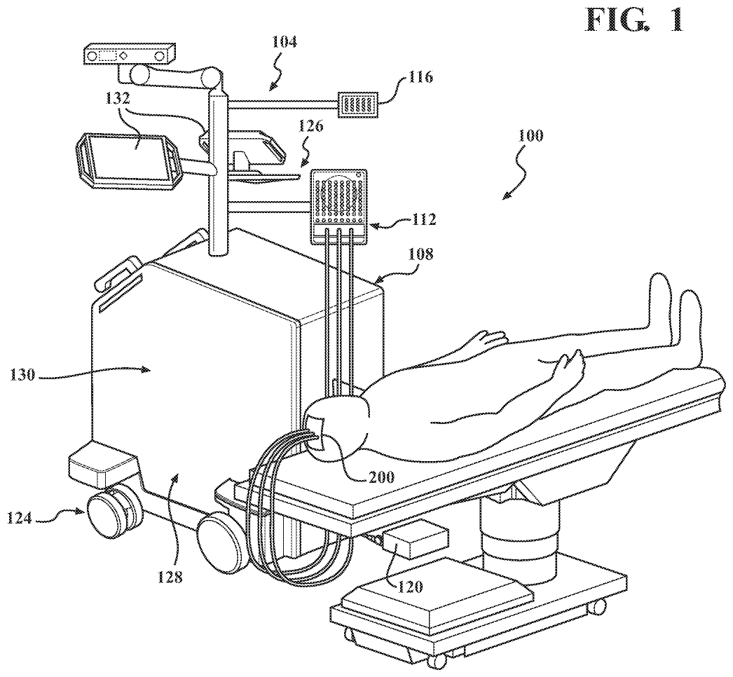

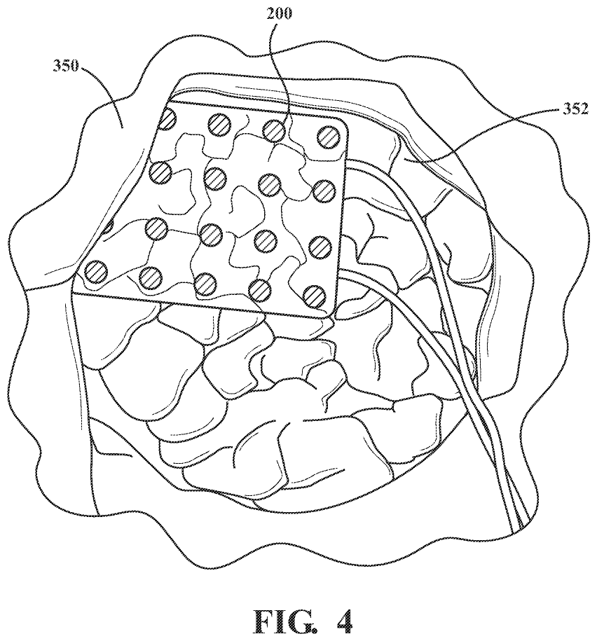

With reference to FIGS. 1 and 4, an exemplary electrode system 100 according to the teachings of the present disclosure is shown. The electrode system 100 includes a surgical navigation system 104, an electromagnetic tracking system 108, an electroencephalography (EEG) amplifier 112, a photic stimulator 116, and a subdural grid electrode array 200.

The surgical navigation system 104 includes a cart assembly 124 that houses a navigation computer 128, an identification device 130 and/or other types of control units. The navigation computer 128 may also be referred to as the navigation controller. A navigation interface is in operative communication with the navigation computer 128. One or more input devices may be used to input information into the navigation computer 128 or otherwise to select/control certain aspects of the navigation computer 128. The navigation interface includes one or more displays 132. Such input devices may include interactive touchscreen displays/menus, a keyboard, a mouse, a microphone (voice-activation), gesture control devices, or the like.

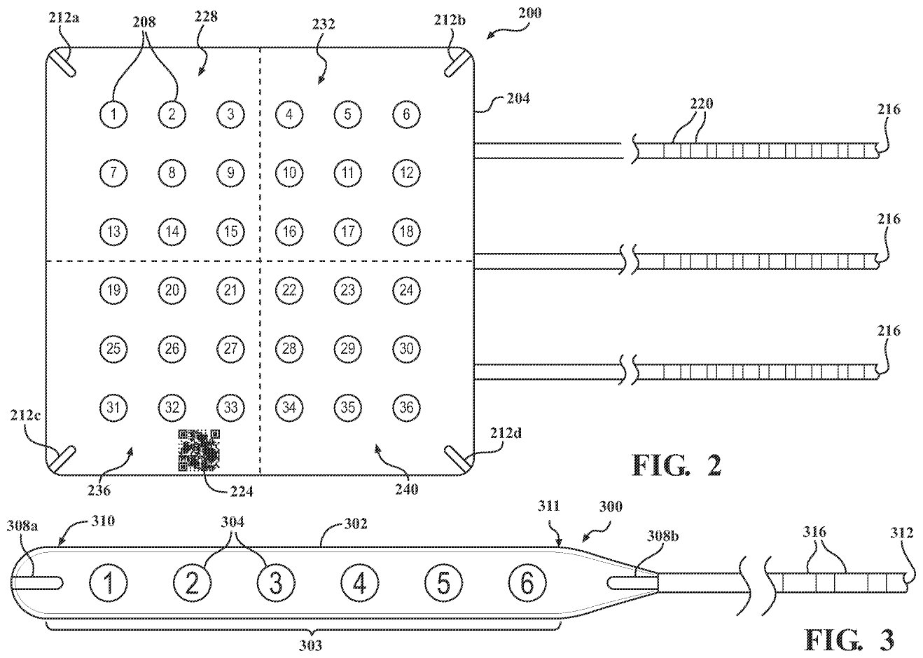

FIG. 2 depicts the subdural electrode array as a subdural grid electrode array 200. The subdural grid electrode array 200 includes a substrate 204, a plurality of electrodes 208 arranged on the substrate 204, tracking devices 212, and tails 216. The plurality of electrodes 208 may measure electrical activity of the brain. The subdural grid electrode array 200 may have various features of the subdural grid electrode array shown and described in U.S. Pat. No. 7,134,919 entitled “Multiple-use, stimulation-accommodating connector,” the contents which are herein incorporated by reference in their entirety. The plurality of electrodes may be arranged on the substrate 204 in M rows by N columns forming an array of electrodes. The tails 216 may include electric contacts 220 that may connect the plurality of electrodes 208 with an EEG amplifier 112 and/or the tracking devices 212 with a sensor interface unit. The number of electric contacts 220 per tail 216 may vary based on the total number of electrodes, the total number of tails 216, and the total number of tracking devices 212. Alternatively, the substrate 204 may include a separate dedicated tail (not shown) for connecting the tracking devices 212 to the sensor interface unit.

With reference to FIG. 4, the substrate 204 is made of non-conductive material and may have any suitable shape, such as a square or rectangular shape. The substrate 204 is configured to be applied directly on a brain of a patient and is conformable to the shape of the brain. The substrate 204 has a profile that is suitable to be slid in between a portion of an intact cranium 350 and a cortex 352 of a patient. For example, a thickness of the substrate 204 may be between 0.3 mm to 0.75 mm. The tails 216 include electric contacts 220. The side of the substrate 204 that is applied to the brain of the patient may include an adherent surface.

In FIG. 3, the subdural electrode array is shown as a subdural strip electrode array 300. The subdural strip electrode array 300 includes a substrate 302, a plurality of electrodes 304, tracking devices 308, and a tail 312 with electric contacts 316. The substrate 302 has a middle portion 303 that has a rectangular shape, a first end 310 that is curved shaped, and a second end 311 that has a triangular shape. The second end 311 connects to the tail 312. The plurality of electrodes 304 in the illustrated configuration are arranged in a single row with six columns for a total of six electrodes 304. However, it is understood that the plurality of electrodes 304 could be arranged in a different manner. For example, the plurality of electrodes 304 may be arranged in two columns with each column having four rows of electrodes 304. Hereinafter the disclosure will discuss the electrode system 100 with respect to the subdural grid electrode array 200 except where explicitly indicated; however, it is understood that the teachings of the disclosure are equally applicable to the subdural strip electrode array 300, any other suitable electrode array, or any combinations of the subdural grid electrode array 200 and the subdural strip electrode array 300. In some examples, the EEG may be performed with multiple electrode arrays including the subdural grid electrode array 200 and multiple subdural strip electrode arrays 300.

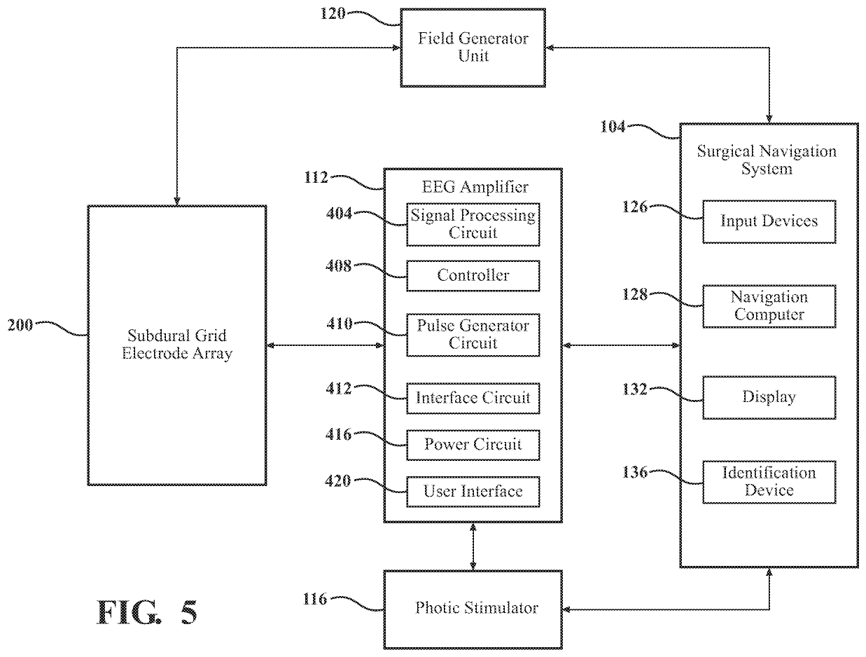

The plurality of electrodes 208 may be configured to provide measurement signals, representative of sensed electrical activity of the brain, to the EEG amplifier 112. The tails 216 may be connected via one or more connectors (not shown) to the EEG amplifier 112 that is configured to process the measurement signals. With reference back to FIG. 5, the EEG amplifier 112 may include one or more signal processing circuits 404, a controller 408, a pulse generator circuit 410, an interface circuit 412, a power circuit 416, and a user interface 420. The EEG amplifier 112 may have various features of the amplifier shown and described in U.S. Pat. No. 7,120,486 entitled “Brain computer interface,” the contents which are herein incorporated by reference in their entirety.

The one or more signal processing circuits 404 may include one or more amplifier circuits, one or more filter circuits, and one or more analog-to-digital converters for receiving and processing the measurement signals from the subdural electrode array. The power circuit 416 may include a power supply with a power input connection for receiving power from a power source and output connections for providing power to the subdural grid electrode array 200. The user interface 420 may be in the form of a display or buttons that allow a medical professional or other person to control the EEG amplifier 112. The user interface 420 may also display notifications or alerts associated with the EEG amplifier 112. The pulse generator circuit 410 may be configured to generate a pulsed waveform in order to deliver electrical stimulation via the subdural grid electrode array 200 to a portion of the brain.

The photic stimulator 116 may be used to provide stimulation during the iEEG procedure. The photic stimulator 116 may have various features of the photic stimulator shown and described in U.S. Pat. No. 6,931,275B2 entitled “System for reduction of undesirable brain wave patterns using selective photic stimulation,” the contents which are herein incorporated by reference in their entirety. The interface circuits 412 may facilitate communication between the EEG amplifier 112 and the surgical navigation system 104. Based on EEG data provided by the EEG amplifier 112, the surgical navigation system 104 may be configured to generate for display waveforms of the EEG data. The surgical navigation system 104 may include an EEG program (i.e., specialized computer software) that processes the EEG data and allows a medical professional to further analyze and refine the EEG data received from the EEG amplifier 112. The surgical navigation system via the EEG program may be configured to control operation of the EEG amplifier 112 and the subdural grid electrode array 200.

The subdural grid electrode array 200 may include a unique identifying component 224, such as a QR code, a bar code, an RFID tag, or a serial read-only memory chip that stores a unique identifier. The identification device 130 may be employed to identify the subdural grid electrode array 200 based on the unique identifying component 224. The identification device 130 may comprise one or more sensors, such as optical sensors, RF sensors, a scanner, a reader, or any other suitable sensor for identifying the unique identifying component 224. For example, in a case where the unique identifying component 224 is a QR code, the identification device 130 may be a camera that is configured to scan and read the QR code.

The electromagnetic tracking system 108 includes a field generator unit 120, the tracking devices 212, a sensor interface unit (not shown), and a sensor control unit (not shown). One example of an electromagnetic tracking system known in the art that may be employed as the electromagnetic tracking system 108 of this disclosure is the Aurora® System made by Northern Digital, Inc. The field generator unit 120 provides a reference point and generates an electromagnetic (EM) field.

The field generator unit 120 may be statically positioned (as shown in FIG. 1) with a longer-range field or may be a handheld device with a shorter-range field. In electromagnetic tracking systems 108, tracking errors may occur when the EM field interacts with ferromagnetic materials or other nearby electronic devices which are prevalent in neurosurgery. In the latter case, field distortion tracking errors would be minimized; however, the field generator unit 120 would be optically tracked in order to sync a reference point.

The tracking devices 212 may include one or more sensor coils that detect the EM field when the subdural grid electrode array 200 is within a field of range of the field generator unit 120. The tracking devices 212 output signals based on the detected EM field that are indicative of a state of the tracking devices 212 relative to the field generator unit 120. As used herein, the state of the tracking devices 212 includes, but is not limited to, data that defines the position and/or orientation of the tracking devices 212 or equivalents/derivatives of the position and/or orientation. The tracking devices 212 may be any suitable electromagnetic tracking element, such as a coil array or the like, arranged on the substrate 204. The tracking devices 212 have a size and profile that make the tracking devices 212 suitable to be slid in a subdural space such that the tracking devices 212 do not disturb or damage dura or brain tissue. The sensor interface unit may amplify and digitize the signals provided by the tracking devices 212. The system control unit determines a state for each of the tracking devices 212 based on a strength of the EM field as evidenced by the signals received from the respective tracking devices 212.

The tracking devices 212 are arranged on the substrate 204 in a predetermined position. As shown in FIG. 2, there are four tracking devices 212 arranged on the subdural grid electrode array 200. A first tracking device 212a is arranged on a first portion 228 of the subdural grid electrode array 200, a second tracking device 212b is arranged in a second portion 232 of the subdural grid electrode array 200, a third tracking device 212c is arranged in a third portion 236 the subdural grid electrode array 200, and a fourth tracking device 212d is arranged in a fourth portion 240 of the subdural grid electrode array 200. While FIG. 2 is described as including four tracking devices 212, any number of tracking devices 212 may be provided. The plurality of electrodes 208 have a first predefined relationship with the first tracking device 212a, a second predefined relationship with the second tracking device 212b, a third predefined relationship with the third tracking device 212c, and a fourth predefined relationship with the fourth tracking device 212d. For example, the predefined relationships may include a distance, a position, an offset, or another suitable spatial relationship of each of the plurality of electrodes 208 relative to each of the tracking devices 212. Based on the state of the tracking devices 212 and the predefined relationships, the surgical navigation system 104 may determine a position for each of the plurality of electrodes 208.

As shown in FIG. 3, the subdural strip electrode array 300 may include two tracking devices 308. A first tracking device 308a is arranged at the first end 310 of the subdural strip electrode array 300 and a second tracking device 308b is arranged at the second end 311 of the subdural strip electrode array 300. The first tracking device 308a of the subdural strip electrode array 300 has a first predetermined relationship with the plurality of electrodes 304 and the second tracking device 308b of the subdural strip electrode array 300 has a second predetermined relationship with the plurality of electrodes 304.

The unique identifying component 224 may be used by the surgical navigation system 104 to identify one or more characteristics of the subdural grid electrode array 200. For example, the surgical navigation system 104 may store a look up table of unique identifiers along with one or more characteristics. For example, the one or more characteristics may be a number of the plurality of electrodes 208, a number of rows and columns of the plurality of electrodes 208, a size of the substrate 204, a label for each of the plurality of electrodes 208, a position for each of the tracking devices 212 relative to the substrate 204, and the predefined relationships for each of the tracking devices 212 relative to each of the plurality of electrodes 208. The surgical navigation system 104 may be configured to determine a position for each of the plurality of electrodes 208 based on a nearest tracking device 212. For example, the surgical navigation system 104 may determine a position for each electrode arranged in the first portion 228 based on the position of the first tracking device 212a, a position for each electrode that is arranged in the second portion 232 based on the position of the second tracking device 212b, a position for each electrode arranged in the third portion 236 based on the position of the third tracking device 212c, and a position for each electrode arranged in the fourth portion 240 based on the position of the fourth tracking device 212d.

In another example, the surgical navigation system 104 may be configured to determine a position for each of the plurality of electrodes 208 based on any one of the tracking devices 212. For example, the surgical navigation system 104 may determine a position for each electrode arranged in the first portion 228 based on the position of the second tracking device 212b, the position of the third tracking device 212c, or the position of the fourth tracking device 212d.

In other examples, the surgical navigation system 104 may be configured to determine a position for each of the plurality of electrodes 208 based on all of the tracking devices 212, based on each position of the tracking devices 212, based on known properties of the substrate 204 and predefined relationships for the tracking devices 212, and predefined relationships for the substrate 204 and associated positions for each of the plurality of electrodes 208. An algorithm can be applied to render the flexing of the substrate 204 and resultant position for each of the plurality of electrodes 208. The flexing of the substrate 204 may be determined based on the position and orientation of the tracking devices 212. For example, a lookup table may associate position and orientation information of the tracking devices 212 with a flexing of the substrate 204. The lookup table may also associate experimentally determined offsets/displacements for each of the plurality of electrodes 208 with the flexing of the substrate 204.

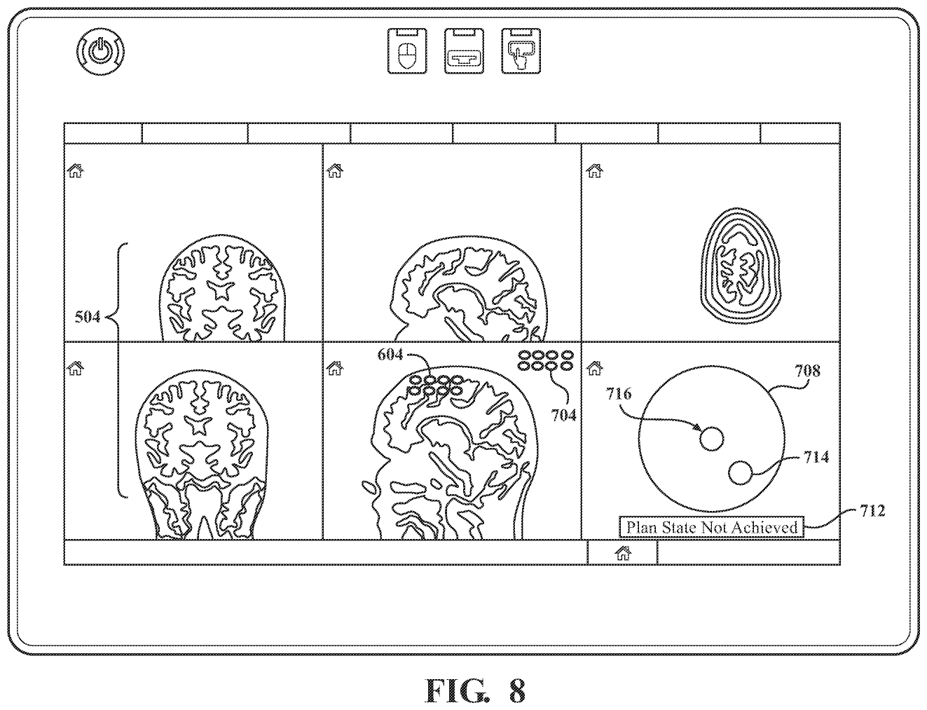

The surgical navigation system 104 may be configured to store one or more pre-operative images of the brain 504. The surgical navigation system 104 may utilize data acquired from the one or more pre-operative images in order to determine a shape or a structure of the brain in which the subdural grid electrode array 200 is applied. The surgical navigation system 104 may utilize the shape or structure of the brain in determining a position for each electrode of the plurality of electrodes 208. In this manner, the surgical navigation system 104 is able to determine a position for each electrode of the plurality of electrodes 304 of the subdural strip electrode array 300 even when the strip bends around the curvature of the brain as shown in FIG. 8.

Any suitable imaging device may be used to provide the pre-operative images of the brain 504. For example, any 2D, 3D or 4D imaging device, such as isocentric fluoroscopy, bi-plane fluoroscopy, ultrasound, computed tomography (CT), multi-slice computed tomography (MSCT), magnetic resonance imaging (MRI), positron emission tomography (PET), optical coherence tomography (OCT). The images may also be obtained and displayed in two, three or four dimensions. In more advanced forms, four-dimensional surface rendering regions of the body may also be achieved by incorporating patient data or other data from an atlas or anatomical model map or from pre-operative image data captured by MM, CT, or echocardiography modalities.

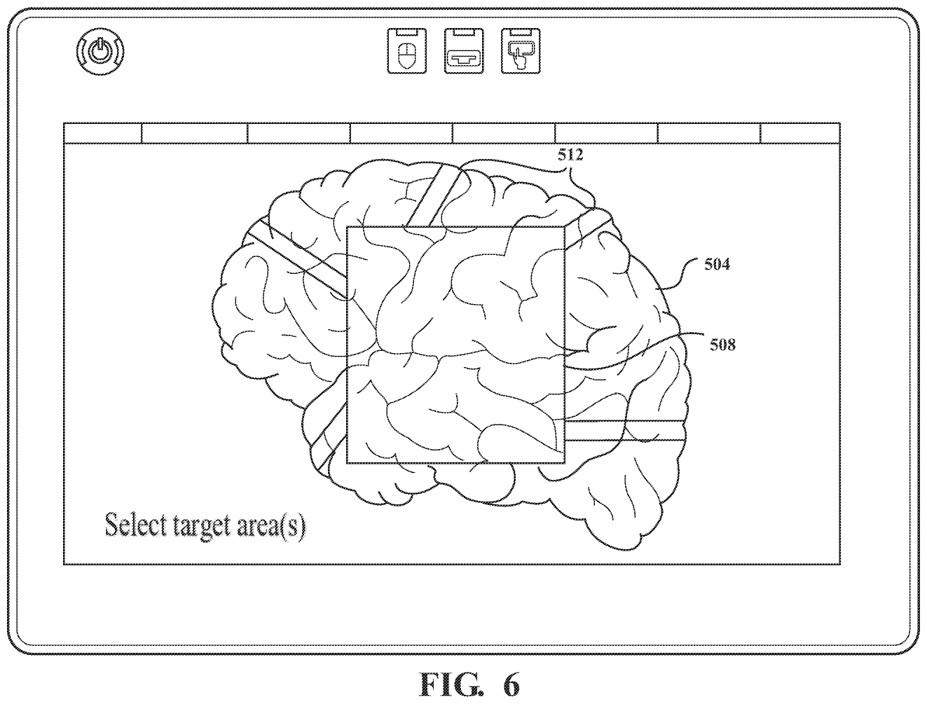

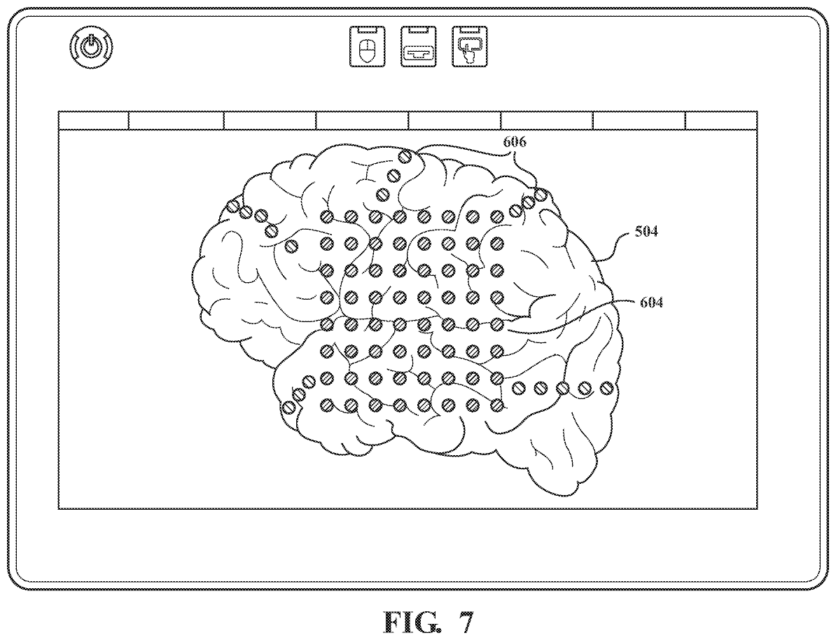

With reference to FIGS. 6 and 7, the surgical navigation system 104 may generate for display the one or more pre-operative images of the brain 504. The surgical navigation system 104 may prompt the user to select one or more target areas 508, 512 of the brain in which the subdural grid electrode array 200 is to be applied. The user may select the one or more target areas 508, 512 of the brain via the touchscreen or the mouse. The one or more target areas 508, 512 refer to the area of interest such as where the medical professional would like the subdural grid electrode array 200 or the subdural strip electrode arrays 300 to be applied.

The surgical navigation system 104 may be configured to store a list of electrode arrays and associated characteristics for each electrode array in the list. The associated characteristics may be a number of the plurality of electrodes 208, a number of rows and columns of the plurality of electrodes 208, a size of the substrate 204 of the subdural grid electrode array 200, a label for each of the plurality of electrodes 208, a location of the tracking device 212 relative to the substrate 204 of the subdural grid electrode array 200, and the predefined relationship between the one or more tracking devices 212 and each of the plurality of electrodes 208. In response to a user selecting a target area, the surgical navigation system 104 may prompt the user to select a subdural electrode array from the list of subdural electrode arrays.

The surgical navigation system 104 may be configured to generate a representation of a virtual electrode arrays 604, 606 corresponding to the type of subdural electrode arrays selected by the user. The virtual electrode arrays 604, 606 may be overlaid onto the selected target area. The surgical navigation system 104 may be configured to allow the user to adjust the state of virtual electrode arrays 604, 606 if the medical professional is not satisfied with the initial state of the virtual electrode arrays 604, 606. The desired state of the subdural grid electrode array 200 and the subdural strip electrode arrays 300 as evidenced by the virtual electrode arrays 604, 606, will be referred to as the planned state from this point on. As shown in FIG. 8, when the EEG is performed with multiple electrode arrays, the surgical navigation system 104 may generate a virtual representation for each of the multiple electrode arrays. Although the disclosure is discussed in terms of one subdural grid electrode array 200, the concepts of this disclosure can be extended to any number of electrode arrays.

The medical professional may register the generator coordinate system to the image coordinate system (i.e., align the generator coordinate system to the image coordinate system). During registration, a coordinate transformation matrix between the generator coordinate system and the image coordinate system is determined. Registration may be performed using any suitable method of registration (point-pair registration, surface registration, etc.).

With reference to FIG. 8, the surgical navigation system 104 may be configured to simultaneously display multiple views of the subdural grid electrode array 200, the virtual electrode array 604, the one or more pre-operative images 504 of the brain, the target area of the brain, and a graphic or alpha-numeric indication of the state of the subdural grid electrode array 200 relative to the planned state. The surgical navigation system 104 is configured to receive and analyze the state signals to associate relative states to the subdural grid electrode array 200. The surgical navigation system 104 may generate a display of the subdural grid electrode array 200 including a real-time state 704 of the subdural grid electrode array 200 overlaid onto the one or more pre-operative images of the brain 504.

The surgical navigation system 104 may be configured to monitor the state of the subdural grid electrode array 200 to determine when the state of the subdural grid electrode array 200 matches the planned state (i.e., the subdural grid electrode array 200 has been applied to the correct location of the brain) based on the location of the tracking devices 212 relative to a location of the tracking devices of the virtual electrode array 604. The surgical navigation system 104 may also confirm that the subdural grid electrode array 200 has been applied to the brain based on a change in the electrical measurements of the plurality of electrodes 208. When the state of the subdural grid electrode array 200 matches the planned state, the surgical navigation system 104 may generate an indication that the planned state has been achieved. The alert or indication may be in the form of an audible alert, a graphic indication 708 or an alpha-numeric indication 712.

The surgical navigation system 104 may selectively determine a position for each of the plurality of electrodes 208 based on the state of each of the tracking devices 212 and the predefined relationships. The surgical navigation system 104 may be configured such that the medical professional is able to predefine a naming convention for each electrode array in a case where multiple electrode arrays are used. The surgical navigation system 104 may also be configured such as to display a name for each electrode array and a label for each electrode overlaid onto the one or more pre-operative images of the brain 504. The medical professional may capture a screenshot of the display 132 including the names and labels that may be stored and retrieved by the surgical navigation system 104. The label for each electrode may be associated with a corresponding channel (i.e., waveform). The medical professional may use the stored images including screen shots to define a resection area based on results of the EEG. For example, the medical professional may select channels of interest which may then be used to identify the resection area based on the location of electrodes associated with the channels of interest.

As shown in FIG. 8, the surgical navigation system 104 may display a moving circle 714 that moves along with the subdural grid electrode array 200. The moving circle 714 is shown on the display relative to a target 716 (e.g., representation of crosshairs or a second circle) on the display, with the target 716 being representative of the planned state. Once the moving circle 714 overlaps the target 716, the state of the subdural grid electrode array 200 matches the planned state.

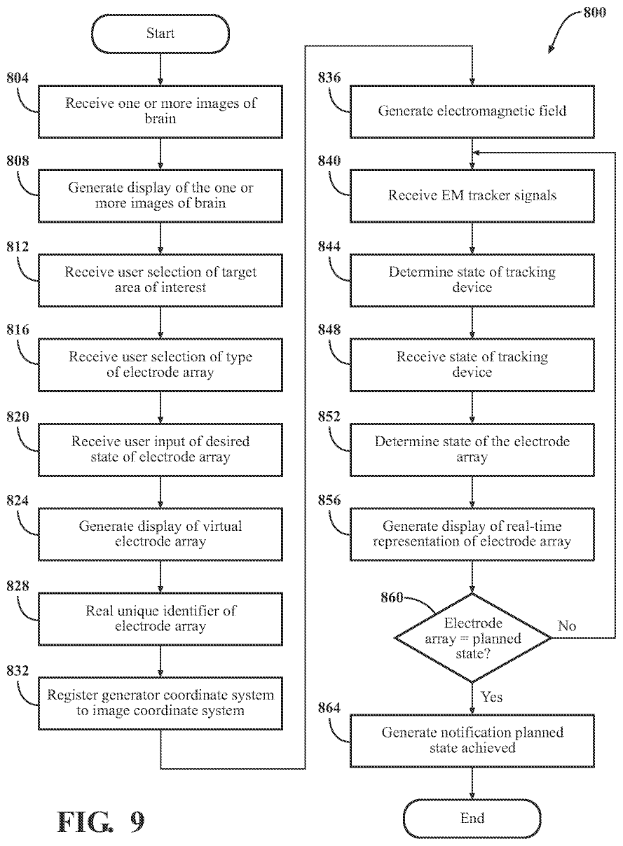

With reference to FIG. 9, an example method 800 of the electrode system 100 will be described. At 804, the surgical navigation system 104 receives and stores one or more pre-operative images of the brain 504 from the imaging device. At 808, the surgical navigation system 104 generates for display the one or more pre-operative images of the brain 504 and the method 800 continues at 812. At 812, the surgical navigation system 104 receives a user selection of a target area of interest. At 816, the surgical navigation system 104 receives a user selection of a type of electrode array and the method 800 continues to 820. At 820, the surgical navigation system 104 receives user input of a desired state of the selected subdural grid electrode array 200. At 824, the surgical navigation system 104 generates a representation of the virtual electrode array 604 for display and the method 800 continues to 828. At 828, surgical navigation system 104 reads and stores the unique identifier associated with the subdural strip electrode array 300.

At 832 the surgical navigation system 104, registers the generator coordinate system to the image coordinate system. At 836, the field generator unit 120 generates an electromagnetic field. At 840, the sensor interface unit receives signals from the EM tracking devices 212. At 844, the sensor control unit determines a state for each of the tracking devices 212. At 848, the surgical navigation system 104 receives the state of each of the tracking devices 212. At 852, the surgical navigation system 104 determines a state of the subdural grid electrode array 200 including a position for each of the plurality of electrodes 208 based on the state of each of the tracking devices 212 and the predefined relationships.

At 856, the surgical navigation system 104 generates a real-time representation of the subdural grid electrode array 200 on the display 132. At 860, the surgical navigation system 104 determines whether the state of the subdural grid electrode array 200 matches the planned state. If so, the method 800 continues to 864; otherwise, the method 800 continues back at 840. At 864, the surgical navigation system 104 generates a notification indicating that the planned state has been achieved. While the method 800 is shown as ending in FIG. 9, the method 800 may be illustrative of one control loop that may be repeated.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the examples is described above as having certain features, any one or more of those features described with respect to any example of the disclosure can be implemented in and/or combined with features of any of the other examples, even if that combination is not explicitly described. In other words, the described examples are not mutually exclusive, and permutations of one or more examples with one another remain within the scope of this disclosure.

Spatial and functional relationships between elements (for example, between controllers, circuit elements, semiconductor layers, etc.) are described using various terms, including “connected,” “engaged,” “coupled,” “adjacent,” “next to,” “on top of,” “above,” “below,” and “disposed.” Unless explicitly described as being “direct,” when a relationship between first and second elements is described in the above disclosure, that relationship can be a direct relationship where no other intervening elements are present between the first and second elements, but can also be an indirect relationship where one or more intervening elements are present (either spatially or functionally) between the first and second elements.

As used herein, the phrase at least one of A, B, and C should be construed to mean a logical (A OR B OR C), using a non-exclusive logical OR, and should not be construed to mean “at least one of A, at least one of B, and at least one of C.” The term subset does not necessarily require a proper subset. In other words, a first subset of a first set may be coextensive with (equal to) the first set.

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

In this application, including the definitions below, the term “controller” may be replaced with the term “circuit.” The term “controller” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The controller may include one or more interface circuits with one or more transceivers. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the WIFI wireless networking standard) and IEEE Standard 802.3-2015 (also known as the ETHERNET wired networking standard). Examples of a WPAN are the BLUETOOTH wireless networking standard from the Bluetooth Special Interest Group and IEEE Standard 802.15.4.

The controller may communicate with other controllers using the interface circuit(s). Although the controller may be depicted in the present disclosure as logically communicating directly with other controllers, in various implementations the controller may actually communicate via a communications system. The communications system may include physical and/or virtual networking equipment such as hubs, switches, routers, gateways and transceivers. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the controller may be distributed among multiple controllers that are connected via the communications system. For example, multiple controllers may implement the same functionality distributed by a load balancing system. In a further example, the functionality of the controller may be split between a server (also known as remote, or cloud) controller and a client (or, user) controller.

Some or all hardware features of a controller may be defined using a language for hardware description, such as IEEE Standard 1364-2005 (commonly called “Verilog”) and IEEE Standard 1076-2008 (commonly called “VHDL”). The hardware description language may be used to manufacture and/or program a hardware circuit. In some implementations, some or all features of a controller may be defined by a language, such as IEEE 1666-2005 (commonly called “SystemC”), that encompasses both code, as described below, and hardware description.

The term code, as used above, may include software, firmware, and/or microcode, and may refer to programs, routines, functions, classes, data structures, and/or objects. The term shared processor circuit encompasses a single processor circuit that executes some or all code from multiple controllers. The term group processor circuit encompasses a processor circuit that, in combination with additional processor circuits, executes some or all code from one or more controllers. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple controllers. The term group memory circuit encompasses a memory circuit that, in combination with additional memories, stores some or all code from one or more controllers.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Swift, Haskell, Go, SQL, R, Lisp, Java®, Fortran, Perl, Pascal, Curl, OCaml, JavaScript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

1. An electrode system for at least one of an intracranial electroencephalogram, an intracranial electroencephalography, and an electrocorticography, the electrode system comprising:
a subdural electrode array including:
a substrate configured to be applied directly on a brain of a patient, the substrate being conformable to a shape of the brain and having a profile suitable to be placed within a subdural space in between a portion of intact cranium and cerebral cortex of the brain;
a first tracking device arranged on the substrate;
a second tracking device arranged on the substrate; and
a plurality of electrodes, arranged on the substrate, configured to sense electrical activity associated with the brain, wherein each of the plurality of electrodes has a first predefined relationship with the first tracking device and a second predefined relationship;
a tracking system operable to (i) determine a first position of the first tracking device relative to a reference point based on a strength of an electromagnetic field relative to the first tracking device and (ii) a second position of the second tracking device relative to the reference point based on the strength of the electromagnetic field relative to the second tracking device; and
a controller configured to selectively determine a position for each of the plurality of electrodes based on at least one of the first position of the first tracking device and a nearest of the first predefined relationship and the second predefined relationship.

2. (canceled)

3. The electrode system of claim 1, wherein the plurality of electrodes is arranged on the substrate in M rows by N columns.

4. The electrode system of claim 1, wherein M is equal to one and N is equal to at least two.

5. The electrode system as claim 1, wherein M is equal to at least two and N is equal to at least two.

6. The electrode system of claim 1, wherein:
the subdural electrode array includes a unique identifier; and
the controller is configured to identify one or more characteristics associated with the subdural electrode array based on the unique identifier.

7. The electrode system of claim 6, wherein the unique identifier is read from at least one of a bar code, a quick response (QR) code, a radio-frequency identification tag, and a serial read-only memory chip.

8. The electrode system of claim 6, wherein the one or more characteristics include at least one of a size of the subdural electrode array, a number of the plurality of electrodes, a number of rows and columns of the plurality of electrodes, a size of the substrate, a label for each of the plurality of electrodes, a location of the first tracking device relative to the substrate, and the first predefined relationship between the first tracking device and each of the plurality of electrodes.

9. The electrode system of claim 8, wherein the controller is configured to store a plurality of different configurations of electrode arrays.

10. The electrode system of claim 9, wherein the controller is configured to receive a user selection of a type of the subdural electrode array from the plurality of different configurations of electrode arrays.

11. The electrode system of claim 10, wherein the controller is configured to receive and store one or more images of the brain.

12. The electrode system of claim 11, wherein the controller is configured to:
generate for display the one or more images of the brain; and
receive a user selection of a target area of the brain in which the subdural electrode array is to be applied.

13. The electrode system of claim 12, wherein the controller is configured to generate a virtual representation of a virtual electrode array, corresponding to the type of the subdural electrode array selected by the user, overlaid onto the selected target area.

14. The electrode system of claim 13, wherein:
the controller is configured to register a first space associated with the brain to a second space associated with the brain; and
wherein the first space is a generator coordinate system and the second space is an image coordinate system.

15. The electrode system of claim 14, wherein the controller is configured to determine when the subdural electrode array has been applied to a correct location of the brain based on a relationship between the selected target area and a real time location of the subdural electrode array.

16. The electrode system of claim 1, further comprising:
an electroencephalography (EEG) amplifier configured to:
receive the sensed electrical activity of the brain via a plurality of measurement signals; and
amplify the plurality of measurement signals, wherein:
the plurality of electrodes are configured to provide the plurality of measurement signals;
the EEG amplifier is configured to provide the amplified plurality of measurement signals to the controller; and
the controller is configured to determine when the subdural electrode array has been applied to the brain based on the amplified plurality of measurement signals.

17. (canceled)

18. (canceled)

19. (canceled)

20. (canceled)

21. A method for tracking a location of a plurality of electrodes of a subdural electrode array for performing at least one of an intracranial electroencephalogram, an intracranial electroencephalography, and an electrocorticography, the subdural electrode array including a substrate with a tracking device and the plurality of electrodes, wherein each of the plurality of electrodes has a predefined relationship with the tracking device, the method comprising:
sliding the subdural electrode array between at least one of:
a portion of an intact cranium and a cortex of a patient; and
on a cortical surface through an opening due to a bone flap;
selectively determining a position of the tracking device relative to a reference point, based on a strength of a generated electromagnetic field, relative to the tracking device; and
determining a position for each of the plurality of electrodes based on the position of the tracking device.

22. The method of claim 21, further comprising determining when the subdural electrode array has been applied to the cortex, wherein determining when the subdural electrode array has been applied to the cortex comprises:
measuring electrical activity of a brain of the patient with the plurality of electrodes; and
determining that the subdural electrode array has been applied to the cortex based on the measured electrical activity.

23. (canceled)

24. (canceled)

25. The method of claim 22, further comprising displaying a virtual representation of a virtual electrode array, wherein displaying the virtual representation of the virtual electrode array includes:
retrieving one or more stored images of a brain of the patient;
receiving a selection of a target area of the brain from the one or more images;
receiving a selection of a type of electrode array from a list of electrode array configurations;
receiving input regarding a position and an orientation of a selected electrode array relative to the target area; and
displaying the selected electrode array overlaid onto the selected target area of the one or more images.

26. (canceled)

27. (canceled)

28. (canceled)

29. (canceled)
